(19) United States
(12) Patent Application Publication
(10) Pub. No.: US 2006/0013890 A1
(43) Pub. Date: Jan. 19, 2006

(54) Dairy-derived anticholesterol immunoglobulin to lower dietary cholesterol in humans

(76) Inventors: Shawn J. Green, Davis, CA (US); Glenn M. Swartz JR., Mount Airy, MD (US)

Correspondence Address: JOHN S. PRATT, ESQ;KILPATRICK STOCKTON, LLP, 1100 PEACHTREE STREET, ATLANTA, GA, 30309, US

(21) Appl. No.: 11/165,601

(22) Filed: Jun. 23, 2005

Related U.S. Application Data
(60) Provisional application No. 60/583,228, filed on Jun. 24, 2004.

(51) Int. Cl. A61K 35/20

(52) U.S. Cl. 424/535

(57) ABSTRACT

The present invention is related to naturally occurring immunoglobulin to cholesterol found in bovine milk that binds to cholesterol, LDL, IDL, and VLDL cholesterol. Milk and dairy-derived products containing immunoglobulin to cholesterol is internally administered to animals including humans. The composition is effective in lowering blood cholesterol and phospholipids by preventing the absorption of dietary sources of cholesterol in the gastrointestinal tract.

### CROSS-REFERENCE TO RELATED APPLICATIONS

This application claims the benefit of U.S. Provisional Patent Application No. 60/583,228 filed Jun. 24, 2004, which is incorporated herein by reference in its entirety.

### FIELD OF THE INVENTION

The present invention relates to compositions and methods for lowering blood cholesterol in animals, including humans, by administering to the animals milk or dairy-derived products containing naturally occurring immunoglobulin to cholesterol. More specifically, the present invention relates to lowering blood cholesterol and phospholipids by preventing the absorption of dietary sources of cholesterol in the gastrointestinal tract of animals. Further, the present invention relates to compositions and methods of producing dairy-derived products that contain anticholesterol activity. In addition, the present invention relates to a methodology for measuring immunoglobulin protein from bovine milk specific for cholesterol, LDL, VLDL, and IDL.

### BACKGROUND OF THE INVENTION

Cholesterol is found in the vast majority of animal cells, serving as an important component of the plasma membrane and other membrane structures. Cholesterol is a precursor of bile acids, the steroid hormones. Cholesterol is not dietary essential, since it is synthesized in the body. Dietary sources of cholesterol are primarily from animal products.

Once cholesterol is ingested, it is absorbed from the gastrointestinal tract into the intestinal lymph. Dietary cholesterol is a mixture of free and esterified cholesterol. Esterified cholesterol is rapidly hydrolyzed and free cholesterol is incorporated into lipid micelles or cholesterol-rich chylomicrons, together with bile acids and hydrolytic products of food fats. Absorption occurs mainly in the upper small intestine. On average, half of the cholesterol ingested in food is absorbed.

After absorption, the free and esterified cholesterol combine with specific apolipoproteins in the plasma to form chylomicrons and VLDL (very low density lipoproteins). These are then distributed to various tissue cells.

Cardiovascular disease is the leading cause of death in the United States. Epidemiological studies have established that elevated total and LDL cholesterol, termed, the “bad cholesterol” are important risk factors for coronary artery disease. In order to avoid and/or treat coronary artery disease, the use of cholesterol-lowering drugs has become a common medical practice. The cholesterol-lowering drug market is more than $10 billion and steadily increasing. In addition, individuals with mildly-elevated cholesterol also turn to foods and nutrients that are reported to have cholesterol lowering benefits and/or effects to improve their cardiovascular health. “Heart healthy” foods contribute many dollars in sales to the cardiovascular health category.

There are several factors that affect plasma cholesterol concentrations. One of these factors is an increase in the amount of cholesterol ingested each day. Blood cholesterol levels are also influenced by both genetic and environmental factors. These include sex, age, diet, body-weight loss or gain, exercise, stress, and a number of pathological conditions.

Atherosclerosis is a disease of the intima of the arteries, especially of the large arteries, that leads to fatty lesions called atheromatous plaques on the inner surfaces of the arteries. Lipid substances, especially cholesterol, begin to deposit from the blood in the proliferating cells on the inner surface of the arteries, forming the atheromatous plaques.

Arteriosclerotic arteries are easily ruptured. Also, the atheromatous plaques of their surface causes blood clots to develop, with resultant thrombus or embolus formation. Almost half of all human beings in the United States and Europe die of arteriosclerosis. Approximately two thirds of these deaths are caused by thrombosis of one or more coronary arteries, and the remaining one third are caused by thrombosis or hemorrhage of vessels in other organs of the body, such as in the brain, which causes stroke.

It has been known for some time that hypercholesterolemia is a risk factor in the development of arteriosclerosis. Treatments directed toward lowering blood cholesterol levels in individuals have included low-cholesterol diets, exercise, and treatment with nonprescription and prescription drugs. While these treatments have shown success in some individuals, others have been unable to benefit from these treatments. For instance, many people are not able to or are unwilling to comply with low-cholesterol diets and exercise programs. Further, diet and exercise are often not sufficient in and of themselves in lowering highly elevated cholesterol levels.

With respect to pharmaceutical treatment of hyperlipidemia and/or hypercholesterolemia, conventional drugs have included niacin and other prescription antilipemic agents, such as lovastatin (Mevacor®) and gemfibrozil (Lopid®). These medications, however, are not well tolerated in some patients due to their associated side effects, which include flushing, abdominal pain, and liver function test elevations.

In recent years, certain unique treatments for hyperlipidemia have been developed. It has been reported that the administration of hyperimmune milk from bovines may be useful in reducing the accumulation of lipids, thereby preventing hypercholesterolemia (see Sharpe, “Cholesterol-Lowering and Blood Pressure Effects of Immune Milk”, Am. J. Clin. Nutr., 1994: 59:929-34). This general method is the subject matter of U.S. Pat. No. 4,636,384 to Stolle et al. The milk is only useful, however, if the milk-producing bovines are brought to a specific state of immunization by means of periodic booster administrations of an antigen or a mixture of antigens. The Sharpe reference suggests relative low dosage levels of globulin protein per day, i.e., 500 mg of IgG/day.

It has been discovered that several animal species, including humans, have naturally occurring antibodies to cholesterol. Alving, C. R. et al., Clin. Immunotherapeutics, 3:409 (1995). While it has been shown that not all animals have these antibodies, such as rabbits and mice, these animals readily gain them upon immunization with high-cholesterol liposomes containing lipid A or with intraperitoneal application of strong irritating adjuvants, such as silicone oil, that may cause recruitment of cholesterol as an antigen from cellular or necrotic debris to inflammatory sites. Alving, C. R. et al., Curr. Topics Microbiol. Immunol., 210:181 (1996). Researchers have developed a vaccine consisting of a protein-free liposome formulation loaded with cholesterol and lipid A as an antigen for inducing anticholesterol antibodies. Swartz, Jr., G. M., et al., Proc. Natl. Acad. Sci. USA, 85:1902 (1988). Studies in rabbits using these cholesterol liposomes resulted in reduced levels of diet-induced hypercholesterolemia. Alving, C. R., et al., J. Lab. Clin. Med., 127(1):40 (1996).

U.S. Patent Application Publication No. US-2003-0223985-A1 (Yoder et al.) discloses that lipid and cholesterol absorption is significantly reduced if globulin protein isolated from sera is included in the diet of animals at certain defined levels, higher than taught by Sharpe. Further, such globulin protein does not have to be derived from hyperimmune milk as is required by the teachings of Sharpe.

A recently abandoned PCT application by Alving, C. R. (PCT/US99/17151) discloses the use of antibodies specific for cholesterol as an oral food supplement for the purpose of lowering serum cholesterol.

Collectively, the above-mentioned references have explicitly stated that the partially purified, purified and/or blood-dried sources of antibodies or immunoglobulin are added to dairy products. In particular, these references do not recognize that bovine milk is a rich and viable source of specific antibodies to cholesterol and cholesterol-rich LDL and VLDL. Further, the above-mentioned references do not disclose non-IgG immunoglobulin to cholesterol in the milk from naive or immunized animals. All previous claims and published disclosures state that antibodies to cholesterol are derived from animal sera or blood plasma and are primarily of, or exclusively limited, to the IgG isotype or class. Furthermore, all previous published claims and disclosures state that sera or blood plasma-derived antibodies to cholesterol must be purified and provided as either a food supplement or delivered through conventional pharmaceutical dosage forms or added to food and dairy products. Furthermore, all previous publications and claims are exclusively limited to sources of antibodies or immunoglobulins from sera and/or blood plasma from naive and/or immunized animals. Previous studies have reported that elevated markers found in sera or blood plasma from an immune response, such as increased levels of IgG, correlates with a reduction in cholesterol.

The present invention is demonstrably superior from both a technological and economical standpoint. Anticholesterol antibodies from approved dairy sources need only to be quantified for certification. In sharp contrast, sera or blood plasma derived sources need to be quantified, isolated and purified. Such isolation and purification procedures are expensive and time-consuming. Once the antibodies are isolated and purified, the activity of the antibodies must be assessed for certification. Further evaluation will be necessary for human consumption of the isolated immunoglobulin fraction, since the source is from pooled sera and blood plasma from non-dairy bovines.

Presently, there are no sources of dairy-derived anticholesterol active sources or products available to address this problem. The use of purified and partially purified globulin proteins from serum and plasma have been previously disclosed. Others skilled in art have dismissed milk as a rich source of anticholesterol antibodies by stating that purified serum-derived anticholesterol would be added to dairy sources.

Evidence has previously been collected in animal studies that support an immunomodulating effect of orally administered plasma proteins; however, there has been no attempt to identify the anticholesterol activity within bovine milk. Further, there have been studies indicating that the administration of milk from bovines challenged with enteric pathogens tends to reduce the level of blood cholesterol in humans. However, previous studies and research have been unable to identify specific anticholesterol antibodies in milk.

Previous studies conducted by the inventors (Dijkstra, J, Swartz, G. M., Raney, J. J., Aniagolu, J, Nacy, C. A., Green, S. J., 1996, Interaction of anticholesterol antibodies with human lipoprotein. Journal of Immunology 157, 2006-2013, Aniagolu, J, Swartz, G. M., Dijlkstra, J, Madsen, J. W., Raney, J. J., Green, S. J, 1995, Analysis of anticholesterol antibodies using hydrophobic membranes. Journal of Immunology Method 182, 85-92) and others have documented the methods for assessing anticholesterol activity, through the use of a well-defined immunoassay that measures binding activity to low density lipoproteins and purified cholesterol. Until now, the specific anticholesterol antibodies and activity in bovine and porcine milk have not been assessed and measured by immunoassay.

What is needed is a composition and method for lowering blood cholesterol in animals, including humans, by administering to the animals milk or dairy-derived products containing naturally occurring immunoglobulin to cholesterol.

### SUMMARY OF THE INVENTION

The present invention provides for the first time a methodology to measure immunoglobulin protein from bovine milk specific for cholesterol, LDL, VLDL, and IDL. According to the present invention, immunoglobulin protein derived from bovine milk which contains specific binding activity to cholesterol, LDL, IDL, and VLDL, and other lipids, contributes to the reduction is serum cholesterol by preventing the absorption of dietary cholesterol into the lymph system and gastrointestinal tract.

The invention comprises the method for identification and use of naturally occurring antibodies to cholesterol found in bovine and goat dairy sources. The term “anticholesterol active” as used herein means antibodies or immunoglobulin to cholesterol that are capable of binding to cholesterol and other cholesterol-rich phospholipid particles including gut derived phospho-rich micelles, blood derived low density lipoproteins including, but not limited to, LDL, VLDL, and IDL. Anticholesterol active milk or dairy products exhibit binding activity to cholesterol and low density lipoproteins.

This invention comprises the authentication and oral administration of anticholesterol active milk, yogurt, cream, ice cream, powdered milk, and use of anticholesterol antibodies with food supplements and additives to prevent the absorption of dietary cholesterol. Elevated levels of antibodies to cholesterol found in the serum of immune animals reportedly lowers blood cholesterol in rabbits and rodents.

The invention also comprises the enrichment of dairy-derived anticholesterol activity in dairy bovines. Anticholesterol antibodies often occurs naturally in bovine milk and can be further elevated by the immunization of animals with monophosphoryl lipid A (a purified lipid from bacteria used as an adjuvant for human and animal vaccines) or other non-specific immunostimulating agent that enhances the non-specific immune response against various infectious diseases.

As part of the invention, the measuring and certification of anticholesterol activity or enriched anticholesterol activity in milk will be determined by an immunoassay for measuring anticholesterol antibodies.

This invention is predicted on the observation that various sources of commercially available milk contain varying amounts of anticholesterol activity. The present invention is the first report of naturally occurring anticholesterol activity or antibodies in milk. This invention comprises the certification of anticholesterol active milk sources and the use of said sources with the intent to lower dietary cholesterol.

Through the use of an immunoassay, it is an object of the present invention to provide a dairy source containing specific immunoglobulin to cholesterol, LDL, IDL, and VLDL for treating animals which will decrease cholesterol and lipid absorption, thereby potentially lowering blood cholesterol levels.

Another object of the present invention is to provide an immunoglobulin protein supplement which is convenient and economical to administer, and which is derived from naive or hyperimmune milk and certified via an immunoassay for detection of specific antibodies to cholesterol, VLDL, IDL, and LDL in any body fluid.

A further object of the present invention is to provide antibodies specific to cholesterol found in dairy products that may render cholesterol-lowering activity by preventing the absorption of dietary cholesterol in the gastrointestinal tract.

Another object of the present invention is to provide an immunoassay or enzyme-linked immunoabsorption assay (ELISA) or other assay to detect, measure, and certify the bio-active presence of anticholesterol antibodies in milk and dairy products. Through the use of an immunoassay, it is an object of the present invention to provide a dairy source containing specific levels of immunoglobulin to cholesterol, LDL, IDL, and VLDL for treating animals which will decrease cholesterol and lipid absorption, thereby potentially lowering blood cholesterol levels.

A further object of the present invention is to demonstrate the presence of anticholesterol activity in bovine milk.

It is yet another object of the present invention to measure, certify and label anticholesterol activity in dairy products.

These and other objects, features and advantages of the present invention will become apparent after a review of the following detailed description of the disclosed embodiments.

### BRIEF DESCRIPTION OF THE DRAWINGS

FIG. 1 illustrates the steps and method of producing the composition for administration to animals in need of lowered blood cholesterol.

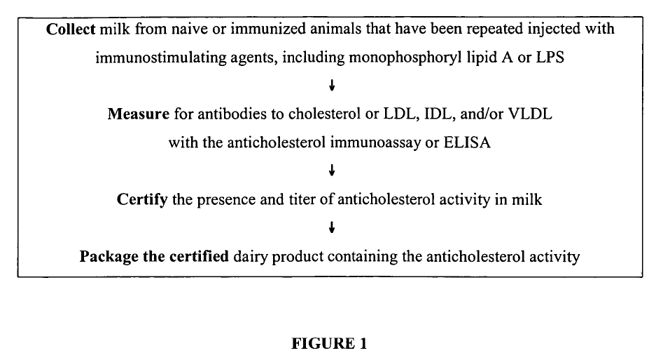

### DETAILED DESCRIPTION OF THE INVENTION

The present invention relates to a new method and composition for lowering blood cholesterol and lipid absorption in animals through the administration of certified dairy-derived anticholesterol antibodies. The invention also relates to a method of measuring anticholesterol activity in dairy milk and related products.

The present invention is in part predicated upon the discovery that the administration of immunoglobulin proteins containing certified anticholesterol activity from animal milk is effective in binding to LDL, VLDL, IDL and other cholesterol-rich phospholipid particles. While sources have reported the existence of antibodies to cholesterol in animal serum and blood plasma, until now there has been no attempt to identify and use the anticholesterol antibodies found in milk, nor the method for measuring the anticholesterol activity in bovine milk.

The present invention discloses the presence of specific immunoglobulin proteins found in bovine milk as measured by a specific method for detecting anticholesterol antibodies. The dairy-derived immunoglobulin of the present invention is capable of binding to cholesterol-rich particles, including LDL, which is associated with hypercholesteremia. While not wishing to be bound by a theory of operation, it is believed that IgM and IgA or an immunoglobulin consisting of gamma heavy or light change that is specific for cholesterol are involved in reducing circulating levels of cholesterol by binding to cholesterol. Other inventions and disclosures are limited to a non-specific pool of sera or blood plasma-derived globulin fraction of IgG as being responsible for lowering cholesterol levels in animals. Further, prior claims of cholesterol lowering by globulin proteins have been unable to measure for specific anticholesterol antibodies in bovine milk.

Unlike other inventions, the composition of this invention is a dairy-based, cholesterol-binding antibody. The anticholesterol containing milk or dairy product is administered to a human or animal, preferably orally. The dairy-based product may be administered as a tablet, in food, or in water.

Using a conventional separation methodology method, the globulin proteins may be further concentrated for administration as a globulin concentrate. The globulin concentrate is stable in water. For purposes of human administration, the dairy product or globulin concentrate may be administered through food, water, juices and other beverages, or other dairy products. Such modes of administration are well known in the art, and have been used for such bacterial products as lactobacillus for many years. The anticholesterol-rich globulin concentrate can also be formulated into a nutraceuticals or pharmaceutical dosage form for oral administration, such as a tablet, capsule, suspension, granules, solution, etc. The pharmaceutical preparations of the present invention are manufactured in a manner which is itself well known in the art. For example, the nutraceuticals or pharmaceutical preparations may be made by means of conventional mixing, granulating, dragee-making, dissolving, lyophilizing processes. The processes to be used will depend ultimately on the physical properties of the active ingredient used. The term “nutraceuticals or pharmaceutically acceptable carrier” is herein defined as a non-toxic carrier that is compatible with the active and inactive ingredients of the formulations of this invention.

Other nutraceuticals or pharmaceutical preparations that can be used orally include push-fit capsules made of gelatin, as well as soft, sealed capsules made of gelatin and a plasticizer such as glycerol or sorbitol. The immunoglobulin protein fraction containing anticholesterol binding activity fraction is placed in the carrier.

The globulin proteins of the present invention are derived from porcine or bovine milk. The milk is obtained through conventional techniques. The milk may be further processed to increase the concentration of the protein component that binds to and blocks cholesterol and lipid absorption.

The isolated immunoglobulin concentrate may also be prepared with one or more appropriate nutraceuticals or pharmaceutical excipients into an oral dosage form for human use.

Detected anticholesterol immunoglobulin in milk from bovine that is naturally produced or potentiated by immunization appears to provide the basis for reducing cholesterol absorption. Naturally-occurring immunoglobulin to cholesterol are elevated in bovines immunized with an immunostimulating agent, such as monophosphoryl lipid A or gram-negative LPS. Hyperimmunization does not appear to be an essential component of the invention.

With respect to humans, the milk product enriched with anticholesterol can be administered through any number of routes, but for convenience, oral administration is preferred through such means as tablets or capsules, or as a supplement, or as a dairy product.

In general, the anticholesterol immunoglobulin proteins should be administered in a concentration in reduce at minimum >4% of total serum cholesterol. Elevated levels of anticholesterol activity in milk can reduce serum cholesterol approximately 5% or greater when administrated daily for extended period of time and possibly more when concentrated and delivered during a meal.

The dairy-derived anticholesterol immunoglobulin proteins may also be administered with certain additives or nutrients, such as carbohydrates, vitamins and minerals. The only requirement is that the additives also be compatible with immunoglobulin concentrate. Such additives can be readily ascertained by those skilled in the art.

This invention comprises that dairy products will be measured, certified, labeled, and used as a dairy product containing anticholesterol activity or anticholesterol antibodies to prevent the absorption of dietary sources of cholesterol. Isolated anticholesterol antibodies from certified milk sources can be used in dairy-derived products, food supplements, additives or alternative foods, including, but not limited to, milk chocolate.

A preferred method of making the anticholesterol active milk and dairy-derived products of the present invention includes, but is not limited or predicated on, the following steps:
1. Identify diary sources of anticholesterol active milk via the immunoassay for antibodies or immunoglobulin described below;
2. Pool certified lots of anticholesterol active milk, based on its bioactivity;
3. Label the dairy source as “anticholesterol active”;
4. Anticholesterol active milk can be used in creams, yogurt, butter, and other dairy products.

A preferred method of authenticating the presence of anticholesterol activity in the milk or dairy-derived products of the present invention, or third party dairy products, includes, but is not limited to, using the anticholesterol immunoassay of the present invention, for purposes of detecting anticholesterol antibody activity. Thereafter, the milk or dairy-derived products can be certified as containing antibodies to cholesterol or as anticholesterol active. In particular, the present invention can be used to provide a milk or dairy source identified or certified as “anticholesterol active” or “containing antibodies to cholesterol and LDL.”

The following examples illustrate but not limit the invention. Thus, the examples are presented with the understanding that modifications may be made and still be within the spirit of the invention.

### EXAMPLE 1

### Anticholesterol Antibodies Are Present in Bovine Milk

The object of this study was to determine the presence of anticholesterol antibody activity in commercially available bovine milk. Using a proprietary ELISA for the detection of anticholesterol antibodies in bovine milk, titers were detected that correlated with the binding of LDL and the lowering of hypercholesteremia in cholesterol-fed rabbits.

The example demonstrates for the first time that anticholesterol antibodies exist in bovine milk and are of the type that can reportedly lower cholesterol and lipid absorption at the micelles or chylomicrons level.

Further, this example demonstrates that the anticholesterol antibodies present in bovine milk are not limited to IgG, and that these naturally occurring anticholesterol antibodies are of the same profile that bind to crystalline cholesterol and LDL.

### EXAMPLE 2

### Method of Identifying, Quantifying and Certifying Anticholesterol Activity in Dairy Products

The object of this study was to determine the presence of specificity of anticholesterol immunoglobulins in commercially available bovine milk. These methods are modifications of methods well-known to those of ordinary skill in the art.

The immunoglobulin is measured by an immunological assay comprised of cholesterol crystals or LDL particles immobilized on plastic plates by which the anticholesterol immunoglobulin in milk, or other dairy-derived fluids, binds to the cholesterol or LDL particles.

### EXAMPLE 3

### Standard for Identifying, Quantifying and Certifying Anticholesterol Activity in Dairy Products

The purpose of this study is to provide a methodology and standard for the dairy industry to produce anticholesterol active dairy products. The standard can be utilized by any producer of dairy products and serve as an authoritative source for identifying, measuring, and certifying anticholesterol activity in dairy products.

The anticholesterol active dairy products of the present invention are purified and isolated from the milk of bovine and porcine sources using techniques well known in the industry. The purified and isolated milk is further measured for the presence and quantity of anticholesterol antibody, through the use of a well-defined immunoassay that measures binding activity to low density lipoproteins and purified cholesterol.

The process for purifying “anticholesterol antibodies” would be adapted from standard methods for the isolation of immunoglobulin or antibodies.

Having described the invention with reference to particular compositions, method for detection, and source of anticholesterol activity, i.e., antibodies to cholesterol, and proposals of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence that is effective to meet the objectives there intended, unless the context specifically indicates to the contrary.

What is claimed is:

1. A composition for lowering cholesterol or reducing atherosclerosis comprising raw or processed milk, colostrum, or serum containing naturally occurring immunoglobulins specific for cholesterol that bind to cholesterol-rich micelles or precursors to LDL in the lumen of the gut or cholesterol-rich low density lipoproteins, including VLDL, IDL, or LDL in the circulation.

2. The composition of claim 1, wherein the immunoglobulins specific for cholesterol are composed of heavy+light chains of the gamma immunoglobulin structure, including the isotypes of IgM and IgA.

3. The composition of claim 1, wherein the total immunoglobulins specific for cholesterol are processed or concentrated to achieve a titer of greater than a reciprocal dilution of 5.

4. The composition of claim 3, wherein the titer is greater than a reciprocal dilution of 5 for the antigen crystalline cholesterol.

5. The composition of claim 3, wherein the titer is a measurement for the amount of immunoglobulins for cholesterol as measured by an ELISA, radioimmunoassay, flow cytometry, or any immuno-based assays used in the detection and measurement of titers for immunoreactivity to cholesterol.

6. A method of lowering cholesterol or reducing atherosclerosis comprising administering a composition comprising raw or processed milk, colostrum, or serum containing naturally occurring immunoglobulins specific for cholesterol that recognize cholesterol-rich chylomicrons in the lumen of the gut or cholesterol-rich low density lipoproteins, including VLDL, IDL, or LDL in the circulation, but not to HDL.

7. The method of claim 6, wherein the raw or processed milk, colostrum, serum is administered orally.

8. The method of claim 6, wherein the raw or processed milk, colostrum, serum is purified to achieve monoclonal or polyclonal immunoglobulins to cholesterol.

9. The method of claim 6, wherein the immunoglobulin is of mammalian source from raw milk, colostrum, serum, or secretion of a lymphocyte or hybridoma isolate in culture.

10. The method of claim 6, wherein the immunoglobulin can be formulated into a dosage form selected from a group consisting of liquid or powder for internal administration.
